(19) United States
(12) Patent Application Publication
(10) Pub. No.: US 2005/0267051 A1
(43) Pub. Date: Dec. 1, 2005

(54) Antineoplastic and anti-HIV conjugates of steroid acid and nucleosides

(76) Inventors: Henry J. Lee, Tallahassee, FL (US); Zhengqing You, Tallahassee, FL (US)

Correspondence Address: MILES & STOCKBRIDGE PC, 1751 PINNACLE DRIVE, SUITE 500, MCLEAN, VA, 22102-3833, US

(21) Appl. No.: 11/015,743

(22) Filed: Dec. 20, 2004

Related U.S. Application Data
(60) Provisional application No. 60/531,178, filed on Dec. 19, 2003.

(51) Int. Cl. A61K 31/7072; C07J 17/00

(52) U.S. Cl. 514/26; 536/6.2

(57) ABSTRACT

A conjugate comprising a nucleoside ester of a steroid acid, said steroid acid having a cyclopentanophenanthrene carbon-carbon skeleton or a homolog thereof and containing up to a maximum of 40 carbon atoms and having at least one carboxylic group covalently bonded thereto, said ester exhibiting at least one activity selected from the group consisting of, (1) antiproliferative or antineoplastic activity induced by apoptosis as measured by activity against H9 cells, or (2) anti-HIV activity as measured by activity against T4 lymphocytes or a pharmaceutically acceptable salt, ester, derivative, metal complex, conjugate or prodrug thereof.

### CROSS-REFERENCE TO RELATED APPLICATIONS

This application claims priority based on Provisional Application Ser. No. 60/531,178 filed Dec. 19, 2003. The entire contents and disclosure of which is incorporated herein by reference.

Some of the work leading to the present invention was funded, at least in part, by NIH grants AM 21627, RR 08111, RR 02660 and AI 20360.

### BACKGROUND OF THE INVENTION

1. Field of the Invention

The present invention relates to novel compounds useful as antineoplastic and/or anti-HIV agents; pharmaceutical compositions comprising the compounds; methods of treatment employing the compounds and packaged articles containing the compounds.

2. Description of the Prior Art

Azidothymidine (AZT), the first clinically approved agent for treatment of acquired immunodeficiency syndrome (AIDS), acts by inhibiting the reverse transcriptase of human immunodeficiency virus (HIV) [Mitsuya, H.; Weinhold, K. J.; Furman, P. A.; St. Clair, M. H.; Nusinoff-Lehrman, S.; Gallo, R. C.; Bolognesi, D.; Barry, D. W.; Broder, S. Proc. Nad. Acad. Sci. U.S.A. 1985, 82,7096; Richman, D. D.; Fischl, M. A.; Grieco, M. H.; Gottlieb, M. S.; Volberding, P. A.; Laskin, O. L.; Leedom, J. M.; Groogman, J. E.; Mildvan, D.; Schooley, R. T.; Jackson, G. G.; Durack, D. T.; Phil, D.; King, D. N. Engl. J Med 1987, 317,185; Mitsuya, H.; Broder, S. Nature (London) 1987, 325, 773]. Although AZT has attained a mainstay status in the treatment of AIDS, its serious dose-related side effects have necessitated ongoing research to find safer and more effective analogs [Fischl, M. A.; Richman, D. D.; Grieco, M. H.; Gottlieb, M. S.; Volberding, P. A.; Laskin, O. L.; Lecdom, J. M.; Groogman, J. E.; Mildvan, D.; Hirsch, M. S.; Jackson, G. G.; Durack, D. T.; Musinoff-Lelirman, S. N. Engl. J Med. 1987, 317, 185].

One promising approach to improve the therapeutic index of AZT has been the prodrug strategy. This approach couples AZT with an acid to form an ester which is designed to gradually release AZT by metabolism. Since AZT has a short plasma half-life due to glucuronidation at the 5′-OH [De Clercq, E. Perspectives for the chemotherapy of AIDS. Anticancer Res. 1987, 7, 1023], and poor cellular penetrating capability [Zimmerman, T. P.; Mahony, W. B.; Prus, K. L. J Biol. Chem. 1987, 26-2,5748], esterification would be expected to increase drug stability against metabolic destruction and improve its cellular bio-availability, thus reducing dose-related toxicity. The most studied AZT prodrugs have been carboxylic esters and phosphoesters. The former includes the use of bicyclams attached to an acid group [Dessolin, J.; Galea, P.; Vlicghe, P.; Chermann, J.-C.; Kraus, J.-L. J Med Chem. 1999, 42, 229], retinoic acids [Aggarwal, S. K.; Gogu, S. R.; Rangan, S. R. S.; Agrawal, K. C. J Med. Chem. 1990, 33, 1505], 1,4-dihydronicotinic acids [Aggarwal, S. K.; Gogu, S. R.; Rangan, S. R. S.; Agrawal, K. C. J Med Chem. 1990, 33, 1505], and steroid acids [Sharma, A. P.; Ollapally, A. P.; Lee, H. J. Antiviral Chem. Chemother. 1993, 4(2), 93]. Some of the latter are derived from salicyl alcohols [Meier, C.; Knispel, T.; De Clercq, E.; Balzarin, J. J Med. Chem. 1999, 42, 1604], ether lipids [Hong, C. I.; Nechaev, A.; Kirisits, A. J.; Vig, R.; West, C. R.; Manouilov, K. K.; Chu, C. K. J Med Chem. 1996, 39, 177], lipophilic glycosides [Namanc, A.; Gouyette, C.; Fillion, M.-P.; Fillion, G.; Huynh-Dinh, T. J Med. Chem. 1992, 35, 3039.

In the design of new steroid conjugates with AZT, four rationales are employed: 1. The use of biologically inactive steroid acids based on the antedrug concept [Lee, H. J.; Soliman, M. R. I. Science, 1982, 215, 989] which are devoid of systemic toxicity. 2. The acids are designed to improve the lipophilicity and bio-avail ability of the resulting prodrugs. 3. Protection of the 5′-OH could increase drug half-life in vivo. 4. Binding of steroid conjugates to transcortin could further improve metabolic stability [Rosner, W. Endocrinol Metab. Clin. North Am. 1991, 20(4), 697].

It is an object of the present invention to provide new steroid acid/AZT conjugates possessing unique therapeutic profiles that enable their use in multiple therapy applications.

### SUMMARY OF THE INVENTION

The above and other objects are achieved by the present invention, one embodiment of which relates to novel conjugates comprising AZT esters of steroid acids, the steroid acids having a cyclopentanophenanthrene carbon-carbon skeleton or a homolog thereof and containing up to a maximum of 40 carbon atoms and having at least one carboxylic group covalently bonded thereto, the ester exhibiting at least one activity selected from the group consisting of, (1) antiproliferative and/or antineoplastic activity induced by apoptosis as measured by activity against H9 cells, or (2) anti-HIV activity as measured by activity against T4 lymphocytes or a pharmaceutically acceptable salt, ester, derivative, metal complex, conjugate or prodrug thereof.

Another embodiment of the invention concerns an antiproliferative or antineoplastic pharmaceutical composition comprising a conjugate described above and a pharmaceutically acceptable carrier therefore.

Still another embodiment of the invention comprises a method of treating a mammalian subject in need of antiproliferative or antineoplastic therapy comprising administering thereto a safe and effective amount of the above-described conjugate.

A further embodiment of the invention relates to articles of manufacture comprising packaging material and a pharmaceutical agent contained within the packaging material, wherein the pharmaceutical agent is effective for the treatment of a subject in need of antiproliferative or antineoplastic therapy, and wherein the packaging material comprises a label which indicates that the pharmaceutical agent can be used for ameliorating the symptoms associated with proliferative growth or neoplasm, and wherein the pharmaceutical agent is one of the above-described conjugates.

Other objects, features and advantages of the present invention will become apparent to those skilled in the art from the following detailed description. It should be understood, however, that the detailed description and specific examples, while indicating preferred embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIGS. 1-4 biological data for compounds of the invention.

### DETAILED DESCRIPTION OF THE INVENTION

The present invention is predicated on the discovery that conjugates comprising certain AZT esters of steroid carboxylic acids, or pharmaceutically acceptable salts, esters, derivatives, metal complexes, conjugates or prodrugs thereof, wherein the steroid acid has a cyclopentanophenanthrene carbon-carbon skeleton or a homolog thereof and contains up to a maximum of 40 carbon atoms and has at least one carboxylic group covalently bonded thereto, and exhibits (1) antiproliferative and/or antineoplastic activity induced by apoptosis as measured by activity against H9 cells, or (2) anti-HIV activity as measured by activity against T4 lymphocytes or a pharmaceutically acceptable salt, ester, derivative, metal complex, conjugate or prodrug thereof.

Although the invention is hereafter specifically illustrated utilizing the steroid acids discussed below, it will be apparent to those skilled in the art that other steroid acids satisfying the above-described criteria for selection may be substituted for either of those illustrated below.

In the following examples, two novel biologically inactive steroid acids (2) and (3): 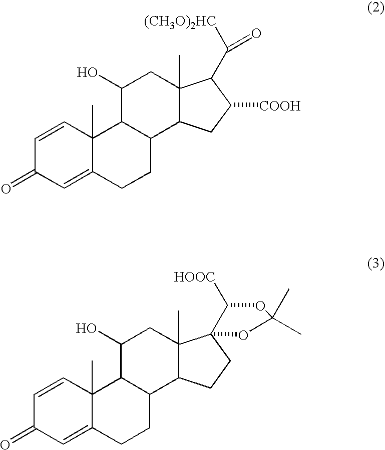
and one commercially available, non-toxic acid (1) cholenic acid) 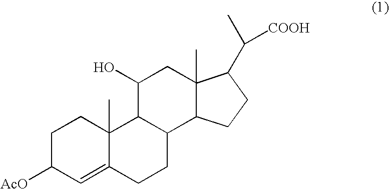
were conjugated with AZT. Preliminary anti-HIV bioassays showed that the two esters (5,6): 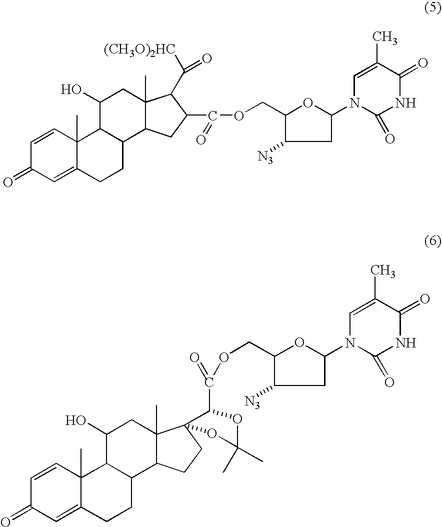
were active against the third (4) 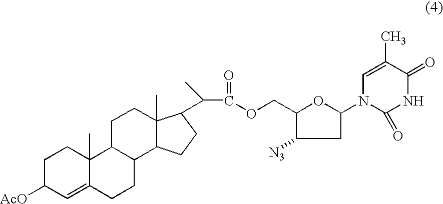
was not. However, the intriguing activity/toxicity profile of the conjugate (4) led to an in-depth evaluation of its anti-proliferation and anti-tumor activities.

### Synthetic Procedure

Structures of Conjugates A-F—In describing the location of groups and substituents, the following numbering system will be employed to conform the numbering of the cyclopentanophenanthrene nucleus to the convention used by the IUPAC or Chemical Abstracts Service:

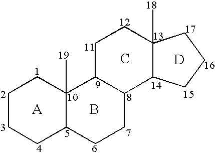

The five- and six-membered rings of the steroid molecule are often designated A, B, C, and D as shown. The term “steroid” as used herein is intended to mean compounds having the aforementioned cyclopentanophenanthrene nucleus.

In these structures, the use of bold and dashed lines to denote particular conformation of groups follows the IUPAC steroid-naming convention. The symbols “.alpha.” and “.beta.” indicate the specific stereochemical configuration of a substituent at an asymmetric carbon atom in a chemical structure as drawn. Thus “.alpha.,” denoted by a broken line, indicates that the group in question is below the general plane of the molecule as drawn, and “.beta.,” denoted by a bold line, indicates that the group at the position in question is above the general plane of the molecule as drawn. 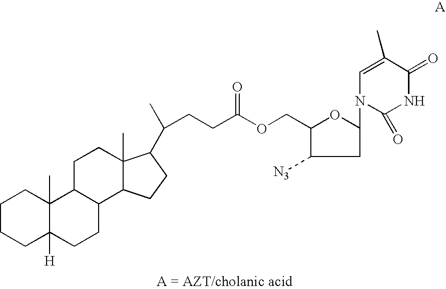 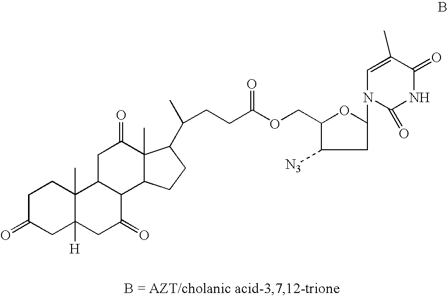 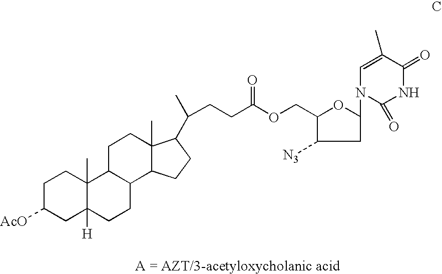
NOTE: A-C are AZT conjugates with three different steroid acids 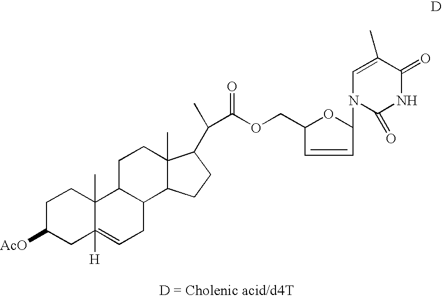 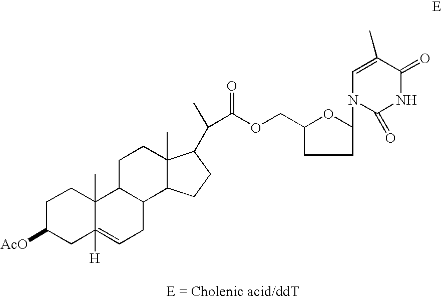 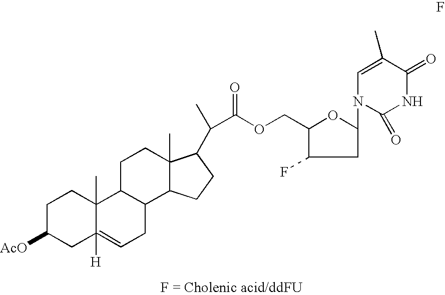
NOTE: D-F are cholenic acid conjugates with three different nucleosides.

A standard procedure for the preparation of compounds A-F is described as follows. A mixture of the steroid acid (1.6 mmol), AZT or other nucleoside (0.82 mmol), DCC (1.6 mmol) and DMAP (0.06 mmol) in dry THF (5 ml) was stirred under a rubber septum for 3 days. The mixture was then filtered, and the solid thoroughly washed with EtOAc. The combined organic solution was condensed, and the resulting residue chromatographed (3:1 benzene/acetone) to yield the major product.

Characterizations of the Steroid-Nucleoside Conjugates

5-Cholanic acid, 5′-(3′-azido-3′-deoxythymidinyl) ester (A) White solid (73%). ¹H NMR (CDCl₃, 270 MHz): 8.92 (1H, br. s, NH), 7.23 (1H, s, H-6″), 6.12 (1H, t, J=6.3 Hz, H-1′), 4.34 (2H, d of AB-quartet, d_{AB}=24.0 Hz, J_{AB}=12.2 Hz, J_{A}=4.4 Hz, J_{B}=3.9 Hz, H-5′), 4.18 (1H, m), 4.09 (1H, m), 1.94 (3H, s, Me of thymidine), 0.92 (3H, d, J=6 Hz, Me of steroid), 0.91 (3H, s, Me of steroid), 0.64 (3H, s, Me of steroid). ¹³C NMR (CDCl₃, 68 MHz): 173.51, 163.34, 149.96, 135.11, 111.25, 85.62, 81.95, 63.25, 60.84, 56.71, 56.04, 43.82, 42.87, 40.64, 40.37, 37.68, 36.00, 35.42, 35.36, 31.14, 31.02, 28.24, 27.57, 27.29, 27.11, 26.62, 24.27, 21.40, 20.91, 18.34, 12.60, 12.11.

5β-Cholanic acid-3,7,12-trione, 5′-(3′-azido-3′-deoxythymidinyl) ester (B) White solid (78%). ¹H NMR (CDCl₃, 270 MHz): 9.09 (1H, br. s, NH), 7.23 (1H, d, J=1.0 Hz, H-6″), 6.09 (1H, t, J=6.3 Hz, H-1′), 4.34 (2H, d of AB-quartet, d_{AB}=25.4 Hz, J_{AB}=12.2 Hz, J_{A}=4.4 Hz, J_{B}=3.9 Hz, H-5′), 4.20 (1H, m), 4.08 (1H, m), 1.93 (3H, s, Me of thymidine), 1.39 (3H, s, Me of steroid), 1.06 (3H, s, Me of steroid), 0.85 (3H, d, J=6.8 Hz, Me of steroid). ¹³C NMR (CDCl₁₃, 68 MHz): 211.73, 208.80, 208.44, 173.33, 163.43, 149.96, 135.26, 111.22, 85.71, 81.86, 63.22, 60.72, 56.90, 51.79, 48.98, 46.81, 45.56, 44.95, 42.78, 38.60, 37.59, 36.43, 36.00, 35.45, 35.30, 31.17, 30.29, 27.60, 25.09, 21.88, 18.61, 12.57, 11.80.

3α-Acetyloxy-5□-Cholanic acid, 5′-(3′-azido-3′-deoxythymidinyl) ester (C) White solid (89%). ¹H NMR (CDCl₃, 270 MHz): 9.34 (1H, br. s, NH), 7.23 (1H, s, H-6″), 6.11 (1H, t, J=6.3 Hz, H-1′), 4.70 (1H, m, H-3), 4.33 (2H, d of AB-quartet, d_{AB}=24.4 Hz, J_{AB}=12.2 Hz, J_{A}=4.4 Hz, J_{B}=3.4 Hz, H-5′), 4.17 (1H, m), 4.08 (1H, m), 1.93 (3H, s, Me of thymidine), 0.91 (3H, s, Me of steroid), 0.90 (3H, d, J=6.5 Hz, Me of steroid), 0.63 (3H, s, Me of steroid).

3β-Acetyloxy-23,24-bisnor-5-cholenic acid, 5′-(3′-deoxy-2′,3′-didehydrothymidinyl) ester (D) White solid (75%). ¹H NMR (CDCl3, 270 MHz): 8.29 (1H, br. s, NH), 6.97 (1H, m, vinyl of thymine), 6.26 (1H, dt, J=5.8, 1.7 Hz, H-2′ or H-3′), 5.92 (1H, ddd, J=5.8, 1.8, 1.2 Hz, H-2′ or H-3′), 5.37 (1H, m, H-6), 5.04 (1H, m, H-1′), 4.53-4.67 (1H, m, H-3), 4.41 (1H, dd, J=12.2, 4.5 Hz, H-5′), 4.20 (1H, dd, J=12.2, 3.4 Hz, H-5′), 2.03 (3H, s, Ac), 1.93 (3H, d, J=1.2 Hz, Me of thymine), 1.21 (3H, d, J=6.7 Hz, H-22), 1.02 (3H, s, Me), 0.69 (3H, s, Me). ¹³C NMR (CDCl₁₃, 75 MHz): 176.40, 170.53, 163.94, 150.76, 139.56, 135.45, 133.08, 127.32, 122.30, 110.99, 89.83, 84.32, 73.83, 64.42, 56.07, 52.47, 49.78, 42.64, 42.32, 39.35, 37.98, 36.85, 36.47, 31.73, 31.67, 27.63, 27.46, 24.27, 21.35, 20.82, 19.20, 17.09, 12.49, 12.01.

3β-Acetyloxy-23,24-bisnor-5-cholenic acid, 5′-(3′-deoxythymidinyl) ester (E) White solid (83%). ¹H NMR (CDCl₁₃, 300 MHz): 9.187 (1H, s, NH), 7.399 (1H, d, J=0.9 Hz, vinyl of thymine), 6.066 (1H, dd, J=6.6, 4.3 Hz, H-1′), 5.354 (1H, m, H-6), 4.589 (1H, m, H-3), 4.20-4.37 (3H, m), 2.023 (3H, s, Ac), 1.940 (3H, s, Me of thymine), 1.231 (3H, d, J=6.8 Hz, H-22), 1.008 (3H, s, Me), 0.685 (3H, s, Me).

3β-Acetyloxy-23,24-bisnor-5-cholenic acid, 5′-(2′,3′-dideoxy-3′-fluorouridinyl)ester (F) White solid (60%). ¹H NMR (CDCl₃, 300 MHz): 8.875 (1H, s, NH), 7.487 (1H, d, J=8.1 Hz, H-6″), 6.297 (1H, dd, J=8.7, 5.4 Hz, H-1′), 5.752 (1H, d, J=8.1 Hz, H-5″), 5.372 (1H, m, H-6), 5.166 (1H, dd, J=53.3, 4.9 Hz, H-3′), 4.602 (1H, m, H-3), 4.498 (1H, dt, J=26.1, 3.7 Hz, H-4′), 4.120-4.394 (3H, m), 2.034 (3H, s, Ac), 1.212 (3H, d, J=6.8 Hz, H-22), 1.021 (3H, s, Me), 0.692 (3H, s, Me).

### Bio-Test Results

Anti-growth activity on H-9 cells is summarized by IC₅₀ data (drug concentration measured by □g/ml needed to inhibit 50% of the cell growth), derived from dose-response curves.

Drug

| | A | B | C | D | E | F |
|---|---|---|---|---|---|---|
| IC₅₀ | 30 | 30 | 4 | 4 | 3 | 20 |

AZT conjugates with steroid acids were synthesized through ester formation of steroid acids with AZT alcohol. Either 1,3-dicyclohexylcarbodiimide (DCC or carbonyldiimidazole (CDI) was used as the coupling agent. Cholenic acid (1) underwent coupling with AZT only under the DCC conditions, while acid (3) yielded the desired product (6) only under the CDI conditions. Although acid (2) could form some desired product (5) under the CDI conditions, the DCC conditions gave a much higher yield of (5). Acid (1) was purchased from Steraloids Inc. (Wilton, N.H.). The two biologically inactive acids (2 and 3) were prepared as shown by Khalil, M. A.; Kwon, T.; Lee, H. J. Current Topics in Med. Chem. 1993, 1,173.

Acid (2) was synthesized by a 4-step procedure starting from enone 9. Nitrile 8 was formed cleanly through a one-step conversion of enone 9 with metal fulminate [You, Z.; Lee, H. J. Tetrahedron Lett. 1996, 3 7(8), 1165]. Mattox rearrangement and methanolysis then yielded methyl ester 7 [You, Z.; Khalil, M. A.; Ko, D.-H.; Lee, J. H. Tetrahedron Lett. 1995, 36(19), 3303] which eventually gave target acid (2) by alkaline hydrolysis. Acid (3) was synthesized diol 11 which in turn was derived from prednisolone using a known literature procedure [Kim, H. P.; Bird, J.; Heiman, A. S.; Hudson, G. F.; Taraporewala, I. B.; Lee, H. J., J. Med Chem. 1987, 30, 2239]. Acetonide 10 was obtained from diol 11 with 2,2-dimethoxypropane and p-toluenesulfonic acid. Alkaline hydrolysis then gave acid (3).

The ester conjugates were subjected to standard in vitro anti-HIV bioassays [see: with T4 lymphocytes (CEM-SS cell line) according to the standard assays described, for example in Bourinbaiar AS, Lee-Huang S. Contraception 1995; 51:319-22 and http://www.iaen.org/files.cgi/7014_Bourinbaiar.pdf. Drug efficacy (EC₅₀) was obtained by adding HIV and AZT conjugate to the cell to measure protection of the drug against HIV-induced celldeath. The cytotoxicity (IC₅₀) data was obtained from adding only the conjugate to the cell to measure cell death caused by the agent. The therapeutic index (TI) is the ratio IC₅₀/EC₅₀. As summarized in Table 1, conjugates (5) and (6) showed similar TI (370 and 184) to that of AZT (>150), while (4) showed no anti-HIV activity. These two active agents appear to have less cytotoxicity and weaker anti-HIV potency than AZT. The inactive agent (4), however, showed high cytotoxicity but no protection against HIV-induced cell death. This intriguing profile of conjugate (4) prompted further evaluations of its growth inhibition in comparison to (5) and (6).

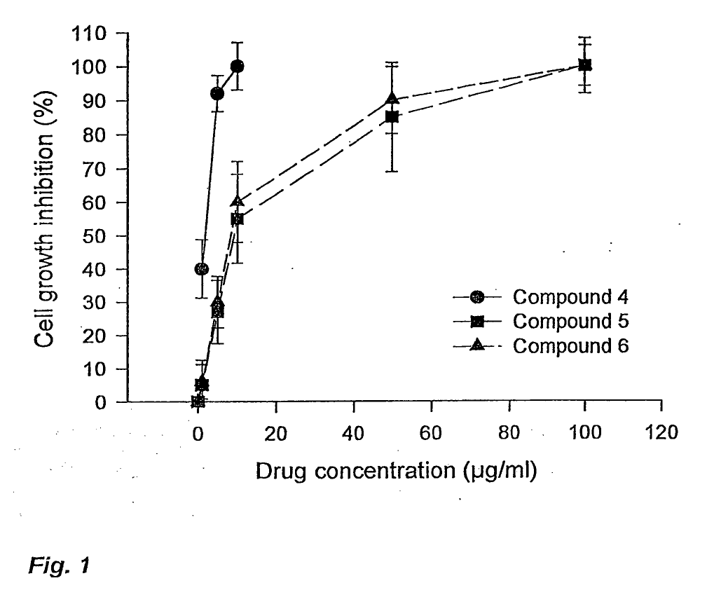

Cell growth inhibitory activity against human lymphoblasts (H9 cells) for compounds (4), (5) and (6) is depicted in FIG. 1. Compound (4) was the most potent with IC50 values of ˜2.5 μg/ml, while the values for the compounds (6) and (5) were estimated to be 10 and 12 μg/ml, respectively. Conjugate (4) was further tested against ddC resistant H9 cells (H9/ddC cells that have reduced thymidine and deoxycytidine kinase activities); HL60, HL60/araC (human promyclocytic leukemic cell lines, parental and araC resistant), H29, H29/FU (human colon adenocarcinomas, parental and FU resistant), MCF7 (human breast carcinoma) and BL5 (Burkitt lymphoma cell line). At 10 μg/ml, it produced 85-100% growth inhibition in all of these human tumor cell lines.

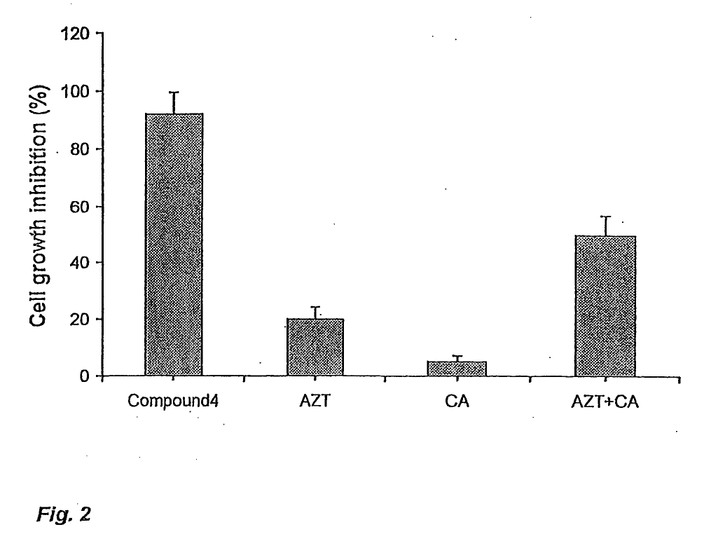

The question of whether the alcohol and acid components within conjugate (4) were responsible for the observed cytotoxicity was addressed by exposure of H9 cells to AZT, cholenic acid, AZT plus cholenic acid, and (4). Although the combination of AZT plus cholenic acid produced more than the additive effect of the two components, conjugate (4) exhibited far greater inhibition and prevented growth of H9 cells almost completely at 5 μg/ml (FIG. 2).

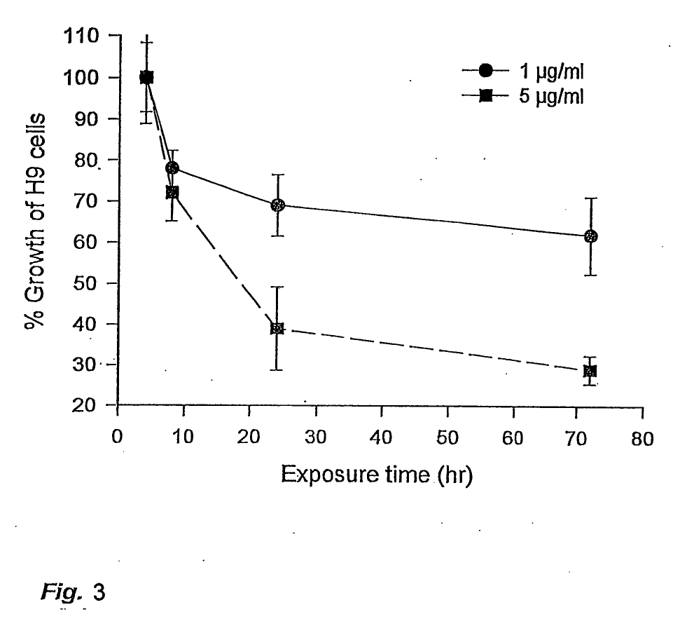

The effect of drug exposure time on cell growth inhibition is shown in FIG. 3. Treatment with conjugate (4) at 1 or 5 μg/ml for 4 hrs did not produce any inhibition while 8 hrs of incubation resulted in 20-25% inhibition. Substantially more cytotoxicity was observed after 24 hrs of exposure at 5 μg/ml, although much longer (72 hrs) exposure only gave a plateau. The data appear to suggest that at least one cell cycle drug exposure is required to cause a significant growth inhibition.

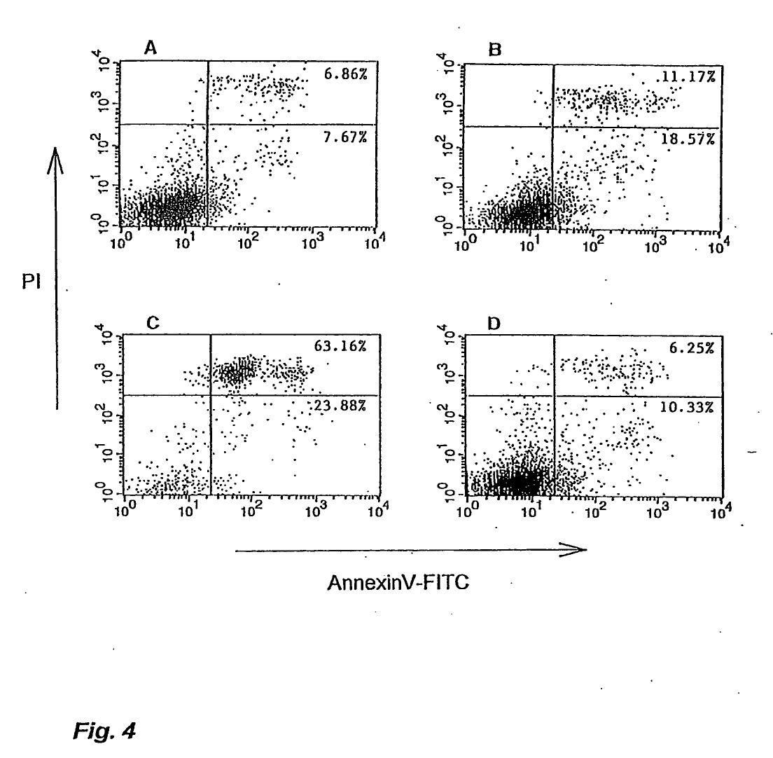

The role of apoptosis in the cellular cytotoxicity of compound (4) was studied and the results are summarized in FIG. 4. In the experiments, fractions of cells in various stages of apoptotic death were assessed according to standard assays [see, e.g., http://www.blackwell-synergy.com/links/doi/10.1046/j.1365-2184.2003.00283.x/abs/; http://64.177.90.157/pfpc/Fluoride_Machalinski_{—}2003.pdf and http://jpet.aspetjournals.org/cgi/content/full/310/1/126 by simultaneous Annexin V and propidium iodide staining using flow cytometry. As indicated, major fractions of H9 tumor cells treated with (4) were in early (FIG. 4C lower right quadrant) or late (FIG. 4C upper right quadrant) stages of apoptotic cell death. In contrast, such effect was significantly less on cells treated with either AZT (FIG. 4B) or cholenic acid (FIG. 4D). Compound (4) at 10 μg/ml appeared to have induced close to 90% of the cells to apoptosis, indicating that the growth inhibition was due to induction of apoptosis and not general necrosis.

For the following examples, prednisolone was purchased from The Upjohn Company, Kalamazoo, Mich. 23,24Bisnor-5-cholenic acid-3β-ol acetate was obtained from Steraloids, Inc., Newport, R.I. All the other chemical reagents were ordered from Aldrich chemical company, Milwaukee, Wis. Preparative flash column chromatography was performed on silica gel (200-425 mesh, Davisil G633), and the solvents used were laboratory grade (Fisher Scientific, Fair Lawn, N.J.). NMR spectra were obtained with a Brucker HX-300 spectrometer and the chemical shifts reported in parts per million (ppm) down field from tetramethylsilane as an internal standard. Elemental analysis was carried out by Galbraith Laboratories, Knoxville, Tenn. Melting points were determined on a Thomas Hoover Capillary Melting Point Apparatus and uncorrected.

### EXAMPLE 1

### 3′-Azido-3′-deoxythymidine-5′-[3β-acetyloxy-22,23-bisnor-5-cholenate] (4)

A mixture of acid 1 (200 mg, 0.51 mmol), AZT (160 mg, 0.59 mmol), DCC (140 mg, 0.68 mmol) and DMAP (14 mg, 0.11 mmol) in dry DMF (2 mL) was stirred under a nitrogen atmosphere for 3 days and then poured in water (100 mL). The resulting aqueous suspension was extracted with ethyl acetate/hexanes (2/1, 300 mL). The organic solution was condensed via rotary evaporation and the resulting residue purified by flash chromatography (3/1 benzene/acetone) to yield, after evaporation of solvent, (4) as a white solid: 66 mg (20%). Proton NMR (DMSO-d6): 11.355 (1H, s, NH), 7.459 (1H, d, J=1 Hz, H-6″), 6.105 (1H, t, J=6.8 Hz, H-1′), 5.326 (1H, m, H-6), 4.43 (2H, m), 4.213 (2H, m, H-5′), 3.958 (1H, m, H-3′), 1.969 (3H, s, Ac), 1.786 (3H, d, J=1 Hz, Me-5″), 1.121 (3H, d, J=6.8 Hz, H-22), 0.963 (3H, s, Me), 0.648 (3H, s, Me). Carbon-13 NMR (75.47 MHz, DMSO-d6): 175.4, 169.71, 163.61, 150.35, 139.46, 136.01, 121.99, 109.77, 83.58, 80.48, 79.16, 73.16, 62.96, 60.15, 55.58, 52.42, 49.34, 41.92, 41.85, 37.65, 36.47, 36.06, 35.54, 31.33, 31.20, 27.32, 26.71, 23.88, 21.04, 20.47, 18.93, 16.83, 12.12, 11.70. Analysis Calculated for C₃₄H₄₇N₅O₇: C64.03%, H7.43%, N10.98%. Found: C63.82%, H7.61%, N10.80%.

### EXAMPLE 2

### 3′-Azido-3′-deoxythymidine-5′-[21,21-dimethoxy-3,20-dioxo-11β-hydroxy-1,4-pregn adiene-16α-carboxylate](5)

Methyl ester 7 was obtained through one-step conversion of enone 9 to hydroxynitrile 8 followed by methanolysis. Ester 7 (800 mg, 1.8 mmol) was dissolved in methanol (10 mL) and aqueous NaOH (4%, 10 mL) added. The mixture was stirred for 20 min and then treated with aqueous HCl (5%) to reach a pH 3. The methanol was removed by rotary evaporation, and the remaining aqueous mixture extracted with ethyl acetate (100 mL). The organic solution was washed with water (20 mL) and then dried over sodium sulfate. The dried solution was condensed and the residue chromatographed (9/1 EtOAc/hexanes) to give acid (2) as a white powder, after evaporation of solvent: 700 mg (86%). Proton NMR (DMSO-d6):12.1 (1H, br s, COOH), 7.31 (1H, d, J=9.8 Hz, H—1), 6.15 (1H, dd, J=9.8, 2.0 Hz, H-2), 5.90 (1H, br s, H-4), 4.63 (1H, m), 4.53 (1H, s, H-21), 4.21 (1H, m), 3.29 (3H, s, MeO), 3.26 (3H, s, MeO), 3.09 (1H, d, J=9.3 Hz, H-17), 1.37 (3H, s, H-19), 0.81 (3H, s, H-18). Acid (2) (200 mg, 0.46 mmol) was mixed with AZT (165 mg, 0.62 mmol), DCC (140 mg, 0.68 mmol) and DMAP (10 mg, 0.08 mmol) in dry DMF (2 mL). The mixture was stirred under a nitrogen atmosphere for 4 days and then poured in water (100 ml). The aqueous mixture was extracted with EtOAc/hexanes (3/1, 300 mL), and the organic layer washed with water (3×80 mL). The organic solution was then condensed and the resulting residue allowed to pass flash column (EtOAc) to yield conjugate 5 as a white solid after evaporation of solvent: 150 mg (47%). Proton NMR (CDCl3): 9.382 (1H, s, NH), 7.160 (1H, d, J=10.1 Hz, H-1), 7.077 (1H, br d, J=1 Hz, H-6″), 6.149 (1H, dd, J=10.1, 1.8 Hz, H-2), 5.959 (1H, t, J=6.4 Hz, H-1′), 5.899 (1 H, br s, H-4), 4.364 (1H, s, H-21), 4.324 (1H, m, H-11), 4.235 (1H, dd, J=12.1, 5.1 Hz, H5′), 4.104 (1H, dd, J=12.1, 4.0 Hz, H-5′), 4.05 (1H, m, H-4′), 3.90 (1H, m, H-3′), 3.485 (1H, m, H-16), 3.267 (3H, s, MeO), 3.256 (3H, s, MeO), 3.157 (1H, d, J=9.0 Hz, 14-17), 1.861 (3H, d, J=1 Hz, Me-5″), 1.329 (3H, s, H-19), 0.827 (3H, s, H-18). Carbon-13 NMR (75.47 MHz, CDCl3): 203.91, 186.54, 175.06, 169.83, 163.82, 156.14, 150.11, 135.47, 127.80, 122.40, 111.44, 103.54, 85.50, 81.47, 69.63, 63.62, 61.17, 60.57, 56.01, 55.19, 54.66

### EXAMPLE 3

### 3′-Azido-3′-deoxythymidine-5′-[11β-hydroxy-17,20α-isopropylidenedioxy-3-oxo-1,4-pregnadiene-21-oate] (6)

Diol 11 was obtained by literature procedure [Kim, H. P.; Bird, J.; Heiman, A. S.; Hudson, G. F.; Taraporewala, I. B.; Lee, H. J., J. Med Chem. 1987, 30, 223 9. It (200 mg, 0.5 mmol) was mixed with 2,2-dimethoxypropane (3 g, 28 mmol) and TsOH.H20 (20 mg, 0.1 mmol) in anhydrous DMF (3 mL), and stirred under a nitrogen atmosphere at 40° C. for 2 days. The resulting mixture was then diluted with EtOAc/hexanes (1/1, 250 mL) and washed with saturated aqueous NaHCO3 (2×10 mL) and water (15 mL). The organic solution was condensed and the resulting residue chromatographed (3/2 EtOAc/hexanes) to give acetonide 10 as a white solid, after evaporation of solvent: 120 mg (54%). Proton NMR (CDCl3): 7.268 (111, d, J=10.1 Hz, H-1), 6.261 (1H, dd, J=10.1, 1.9 Hz, H-2), 6.006 (1H, br s, H-4), 4.567 (1H, s, H-20), 4.445 (1H, in, H-11), 3.774 (3H, s, MeO), 1.495 (3H, s, Me), 1.440 (3H, s, Me), 1.368 (3H, s, Me), 0.945 (3H, s, H-18). Acid 3 was obtained, as a white solid, from acetonide 10 with the procedure for preparation of acid (2), and a yield of 75% achieved. This acid (3) (120 mg, 0.28 nmol) was mixed with CDT (100 mg, 0.61 mmol) in anhydrous DMF (1.5 mL) and stirred under a nitrogen atmosphere for 20 min. AZT (130 mg, 0.49 mmol) was then added. Stirring continued (N₂) for 3 days and the mixture was poured in water (70 mL). The aqueous mixture was extracted with EtOAc/hexanes (2/1, 150 mL. The organic solution was condensed and the residue chromatographed (EtOAc) to yield conjugate (6) as a white solid, after evaporation of solvent: 95 mg (52%). Proton NMR (CDCl3): 9.456 (1H, s, NH), 7.187 (1H, d, J=10. 1 Hz, H-1), 7.084 (1H, br d, J=1 Hz, H-6″), 6.153 (H-1, dd, J=10.1, 1.7 Hz, H-2), 5.925 (1H, t, J=6.4 Hz, H-1′), 5.902 (1H, br s, H-4), 4.464 (1H, s, H-20), 4.40 (1H, H-11), 4.31 (2H, m, H-5′), 4.103 (1H, m, H-4′), 3.98 (1H, m, 0.954 (3H, s, H-18). Carbon-13 NMR (75.47 MHz, CDCl3): 186.64, 170.82, 170.35, 163.94, 156.42, 150.08, 135.69, 128.28, 122.27, 111.54, 111.23, 94.79, 86.16, 81.48, 77.54, 69.98, 63.83, 60.54, 55.31, 50.80, 46.22, 44.10, 39, 25, 37.16, 33.57, 31.95, 31.45, 27.79, 27.36, 23.71, 20.97, 17.84, 12.53. Analysis Calculated for C₃₄H₄₃N₅O₉/0.5H₂O: C60.52%, H6.57%, N10.38%. Found: C60.82%, H6.77%, N9.94%.

### EXAMPLE 4

The anti-HIV assay was carried out with T4 lymphocytes (CEM-SS cell line) through the following standard procedure. Agent is dissolved in dimethyl sulfoxide and diluted 1:100 in cell culture medium before preparing serial half-log 10 dilutions. T4 lymphocytes are added and after a brief interval HIV-1 is added, resulting in a 1:200 final dilution of the compound. Uninfected cells with the compound serve as a toxicity control, and infected and uninfected cells without the compound serve basic controls. Cultures are incubated at 37° C. in a 5% carbon dioxide atmosphere for 6 days. The tetrazolium salt, XTT, is added to all wells, and the cultures are incubated to allow formazan color development. Individual wells are analyzed spectrophotometrically to quantitate formazan production, and in addition are viewed microscopically for detection of viable cells and confirmation of protective activity.

Drug-treated virus-infected cells are compared with drug-treated non-infected cells and with other appropriate controls (untreated infected and untreated non-infected cells, drug-containing wells without cells, etc.) on the same plate. Data are reviewed in comparison with other tests done at the same time and a determination concerning activity is made (Table 1).

TABLE 1
Anti-HIV activity and cytotoxicity in CEM-SS cell line^{a}

| Drug | EC₅₀^{b}(M) | IC₅₀^{c}(M) | TI(IC₅₀/EC₅₀) |
|---|---|---|---|
| 4 | inactive | 4.7 × 10⁻⁶ | |
| 5 | 3.5 × 10⁻⁸ | 1.3 × 1O⁻⁵ | 370 |
| 6 | 6.5 × 10⁻⁸ | 1.2 × 10⁻⁵ | 184 |
| AZT | 7.0 × 10⁻⁹ | >1 × 1O⁻⁶ | >150 |

^{a}Numbers derived by averaging two repeat experiments
^{b}50% effective concentration against HIV cytopathic effects
^{c}50% inhibitory concentration for cell growth

Growth inhibition assays were performed in triplicate by seeding 5×10⁴ Cells/ml in 24-well plates (Costar). Test compounds were added, and the trypan blue-excluding cells were counted after 72 hrs of incubation at 37° C. IC₅₀ values were calculated from growth inhibition curves as described in Agarwal, R. P., and Mian, A. Biochem. Pharmacol. 1991, 42, 905 and Agarwal, Ram P., Han, Tieran, Fernandez, Manilyn. Biochemical and Biophysical Research com in un ica lions. 1999, 262, 657. See FIGS. 3-5.

Apoptosis experiments were carried out by treating H9 Cells (IX106) with compound 4 (10 μg/ml), AZT (10 μg/ml) or cholenic acid (10 μg/ml) for 48 hrs. Cells were collected by centrifugation, washed with growth medium, and stained with Annexin V-FITC and propidium iodide (apoptosis dilution kit; Sigma), according to the manufacturer's protocol, and the fluorescence was analyzed via flow cytometry. See FIG. 4.

The methods of the present invention comprises administering to a mammal requiring antiproliferative, anti-HIV or antineoplastic therapy an effective amount of one or more of the compounds of the invention. Administration may be accomplished either therapeutically or prophylactically by means of pharmaceutical compositions which are prepared by techniques well known in the pharmaceutical sciences.

While the compounds of the invention are preferably administered orally or intrarectally, they may also be administered by a variety of other routes such as transdermally, subcutaneously, intranasally, intramuscularly and intravenously.

The present invention is also directed to pharmaceutical compositions which include at least one compound as described above in association with one or more pharmaceutically acceptable diluents, excipients or carriers therefor.

In making the pharmaceutical compositions of the present invention, one or more compounds will usually be mixed with, diluted by or enclosed within a carrier which may be in the form of a capsule, sachet, paper or other container. When the carrier serves as a diluent, it may be a solid, semi-solid or liquid material which acts as a vehicle, excipient or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 60% by weight of active compound, soft and hard gelatin capsules, suppositories, sterile injectable solutions and sterile packaged powders.

Some examples of suitable carriers, excipients and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, methyl cellulose, methyl- and propyl-hydroxybenzoates, talc, magnesium stearate and mineral oil. The formulations can additionally include lubricating agents, wetting agents, emulsifying and suspending agents, preserving agents, sweetening agents or flavoring agents.

The compositions of the invention may be formulated so as to provide rapid, sustained or delayed release of the active ingredient after administration to the patient by employing procedures well known in the art.

The dose of the compound is that amount effective to prevent occurrence of the symptoms of the disease or to treat some symptoms of the disease from which the patient suffers. By “effective amount,” “therapeutic amount” or “effective dose” is meant that amount sufficient to elicit the desired pharmacological or therapeutic effects, thus resulting in effective prevention or treatment of the disease. Prevention of the disease is manifested by a prolonging or delaying of the onset of the symptoms of the disease. Treatment of the disease is manifested by a decrease in the symptoms associated with the disease or an amelioration of the recurrence of the symptoms of the disease.

The effective dose may vary, depending upon factors such as the condition of the patient, the severity of the symptoms of the disease and the manner in which the pharmaceutical composition is administered.

The compositions are formulated, preferably in a unit dosage form, such that each dosage contains an effective amount of the drug, e.g., the amounts normally employed when compounding anti-HIV pharmaceutical compositions. The term “unit dosage form” refers to physically discrete units suitable as unitary dosages for human subjects and other mammals, each unit containing a predetermined quantity of active material calculated to produce the desired therapeutic effect, in association with one or more of the above-described suitable pharmaceutical diluents, excipients or carriers.

The compounds are effective over a wide dosage range in treating the above disease states, typically the same as currently used in dosing conventional anti-HIV agents. Thus, the term “effective amount” refers to a dosage range of approximately 0.02 to approximately 10.0 grams of a compound of the invention per day are useful in the treatment or prevention of retroviral infection, such as HIV infection, AIDS or AIDS-related complex (ARC), with oral doses 2 to 5 times higher. For example, HIV infection can be treated by administration of from about 0.1 to about 100 milligrams of compound per kilogram of body weight from one to four times per day. In one embodiment, dosages of about 100 to about 400 milligrams of compound are administered orally every six hours to a subject. The specific dosage level and frequency for any particular subject will be varied and will depend upon a variety of factors, including the activity of the specific compound the metabolic stability and length of action of that compound, the age, body weight, general health, sex, and diet of the subject, mode of administration, rate of excretion, drug combination, and severity of the particular condition.

The compounds of Formula I can be administered in combination with other agents useful in the treatment of HIV infection, AIDS or ARC. For example, the compound of the invention can be administered in combination with effective amounts of an antiviral, immunomodulator, anti-infective, or vaccine. The compound of the invention can be administered prior to, during, or after a period of actual or potential exposure to retrovirus, such as HIV. However, it will be understood that the amount of compound actually administered will be determined by a physician in light of the relevant circumstances, including (1) the condition to be treated, (2) the choice of compound to be administered, (3) the chosen route of administration, (4) the age, weight and response of the individual patient, and (5) the severity of the patient's symptoms. Therefore, the above dosage ranges are not intended to limit the scope of the invention in any way.

The compositions are formulated, preferably in a unit dosage form, such that each dosage contains an effective amount of the drug, e.g., the amounts normally employed when compounding antineoplastic pharmaceutical compositions. The term “unit dosage form” refers to physically discrete units suitable as unitary dosages for human subjects and other mammals, each unit containing a predetermined quantity of active material calculated to produce the desired therapeutic effect, in association with one or more of the above-described suitable pharmaceutical diluents, excipients or carriers.

The compounds are effective over a wide dosage range in treating the above disease states, typically the same as currently used in dosing conventional antineoplastic agents. Thus, the term “effective amount” refers to a dosage range of from about 50 to about 500 mg/kg, preferably from about 50 to about 250 mg/kg, the frequency of administration and duration of treatment being dependent upon the type and nature of the animal and disorder treated.

The compounds of Formula I can be administered in combination with other agents useful in the treatment of neoplasms. For example, the compound of the invention can be administered in combination with effective amounts of conventionally employed antineoplastics. The compound of the invention can be administered prior to, during, or after a period of disease. However, it will be understood that the amount of compound actually administered will be determined by a physician in light of the relevant circumstances, including (1) the condition to be treated, (2) the choice of compound to be administered, (3) the chosen route of administration, (4) the age, weight and response of the individual patient, and (5) the severity of the patient's symptoms. Therefore, the above dosage ranges are not intended to limit the scope of the invention in any way.

From the foregoing description, various modifications and changes in the composition and method will occur to those skilled in the art. All such modifications coming within the scope of the appended claims are intended to be included therein. The entire disclosures and contents of each and all references cited and discussed herein are expressly incorporated herein by reference. All percentages expressed herein are by weight unless otherwise indicated.

1. A conjugate comprising a nucleoside ester of a steroid acid, said steroid acid having a cyclopentanophenanthrene carbon-carbon skeleton or a homolog thereof and containing up to a maximum of 40 carbon atoms and having at least one carboxylic group covalently bonded thereto, said ester exhibiting at least one activity selected from the group consisting of, (1) antiproliferative or antineoplastic activity induced by apoptosis as measured by activity against H9 cells, or (2) anti-HIV activity as measured by activity against T4 lymphocytes or a pharmaceutically acceptable salt, ester, derivative, metal complex, conjugate or prodrug thereof.

2. A conjugate of claim 1 wherein said carboxylic group is covalently bonded to the 15-, 16- or 17-C position of the cyclopentano moiety of said steroid acid.

3. A conjugate of claim 2 wherein said carboxyl group comprises —CH₂)ₙ—COOH, wherein n=0-5.

4. An ester of claim 1 having the formula:

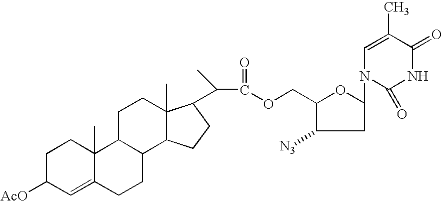

5. An ester of claim 1 having the formula:

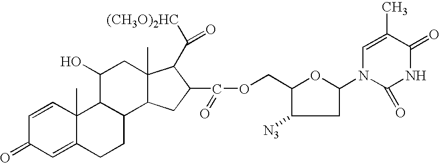

6. An ester of claim 1 having the formula:

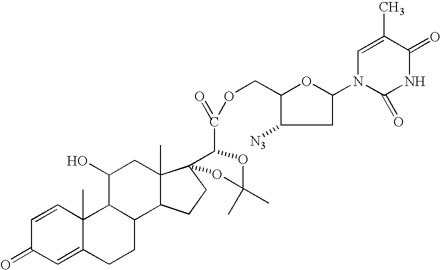

7. An ester of claim 1 having the formula:

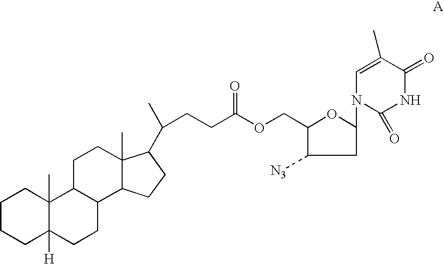

8. An ester of claim 1 having the formula:

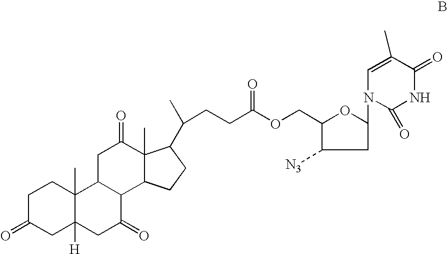

9. An ester of claim 1 having the formula:

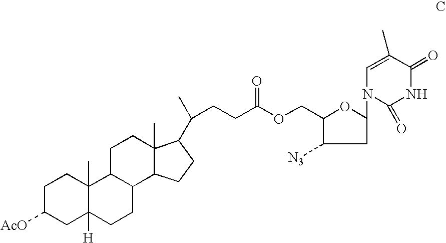

10. An ester of claim 1 having the formula:

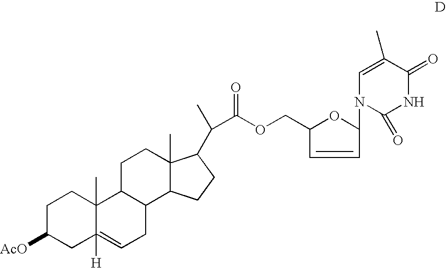

11. An ester of claim 1 having the formula:

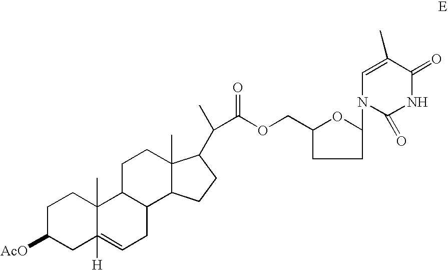

12. An ester of claim 1 having the formula:

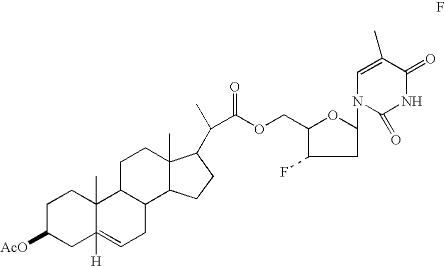

13. An antiproliferative or antineoplastic pharmaceutical composition comprising a conjugate of claim 1 and a pharmaceutically acceptable carrier therefore.

14. An anti-HIV pharmaceutical composition comprising comprising a conjugate of claim 1 and a pharmaceutically acceptable carrier therefore.

15. A method of treating a mammalian subject in need of antiproliferative or antineoplastic therapy comprising administering thereto a safe and effective amount of a conjugate of claim 1.

16. A method of treating a mammalian subject in need of anti-HIV therapy comprising administering thereto a safe and effective amount of a conjugate of claim

17. A steroid acid having the structure:

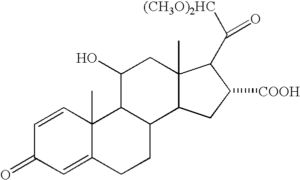

18. A steroid acid having the formula:

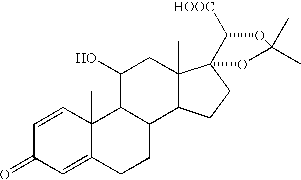

19. An article of manufacture comprising packaging material and a pharmaceutical agent contained within said packaging material, wherein said pharmaceutical agent is effective for the treatment of a subject in need of antiproliferative or antineoplastic therapy, and wherein said packaging material comprises a label which indicates that said pharmaceutical agent can be used for ameliorating the symptoms associated with proliferative growth or neoplasm, and wherein said pharmaceutical agent is a conjugate of claim 1.

20. An article of manufacture comprising packaging material and a pharmaceutical agent contained within said packaging material, wherein said pharmaceutical agent is effective for the treatment of a subject suffering from HIV infection, and wherein said packaging material comprises a label which indicates that said pharmaceutical agent can be used for ameliorating the symptoms associated with HIV infection, and wherein said pharmaceutical agent is a conjugate of claim 1.
